(19) United States
(12) Patent Application Publication
(10) Pub. No.: US 2022/0218737 A1
(43) Pub. Date: Jul. 14, 2022

(54) COMPOSITION COMPRISING INULIN AND PROTEIN

(71) Applicant: Cosucra Groupe Warcoing S.A., Warcoing (BE)

(72) Inventors: Heidi Jacobs, Kruisem (BE); Jérome Salles, Beauregard.I'Evêque (FR); Stéphane Walrand, Aydat (FR); Cathy Signorot, Mézières-en-Brenne (FR)

(73) Assignee: Cosucra Groupe Warcoing S.A., Warcoing (BE)

(21) Appl. No.: 17/610,207

(22) PCT Filed: May 22, 2020
(86) PCT No.: PCT/EP2020/064251

§ 371 Date: Nov. 10, 2021

(30) Foreign Application Priority Data
May 24, 2019 (EP) 19176385.3

(51) Int. Cl. A61K 31/733; A61K 38/16; A61K 35/20; A61K 9/00; A61P 21/06; A23L 33/00; A23L 33/21; A23L 33/19; A23L 33/185; A23L 2/66

(52) U.S. Cl. CPC A61K 31/733; A61K 38/168; A61K 35/20; A61K 9/0053; A61P 21/06; A23V 2002/00; A23L 33/21; A23L 33/19; A23L 33/185; A23L 2/66; A23L 33/40

(57) ABSTRACT

The present invention relates to compositions comprising inulin and protein for reducing loss of muscle mass. The present invention also relates to a composition comprising inulin and protein, wherein said the ratio of said inulin to said protein is between 5/95 to 75/25 by weight. The present invention therefore also concerns the use of a composition comprising inulin and protein for the preparation of a food or a medicament.

### FIELD OF THE INVENTION

The present invention relates to compositions comprising fiber and protein for reducing loss of muscle mass. The present invention also relates to a composition comprising soluble fiber and protein, wherein the ratio of said soluble fiber to said protein is between 5/95 to 75/25 by weight. The present invention therefore also concerns the use of a composition comprising soluble fiber and protein for the preparation of a food or a medicament.

### BACKGROUND OF THE INVENTION

Loss of muscle mass may happen in different situations such as physical inactivity, chronic diseases like diabetes, obesity, sarcopenia, . . .

Loss of muscle mass is related to the net protein balance. Net protein balance (NPB) is defined as muscle protein synthesis (MPS) minus muscle protein breakdown (MPB), or NPB=MPS−MPB. Thus, a significant rise in skeletal MPS (anabolism) and/or reduction in MPB (catabolism), such that NPB remains positive can result in increased skeletal muscle mass accretion. Conversely, a negative NPB, arising from a reduction in MPS and/or increase in MPB, will result in a loss of muscle mass. It has clearly been demonstrated that an acute bout of heavy resistance exercise, intermittent exercise of repeated short, high intensity bouts stimulates a significant increase in MPS.

Aging is characterized by a significant change in body composition, in particular a decrease in lean body mass. The latter phenomenon, well-known as sarcopenia, is the decline in skeletal muscle mass and function with age. The progressive loss of muscle mass with age can reach up to 11 kg between 30 and 80 years and lead to many functional and metabolic consequences. It is accompanied by a reduction in strength and, consequently, contributes to the loss of autonomy and the risk of falling in older people. The muscle is a reservoir of amino acids that can be mobilized in a situation of need. In fact, sarcopenia contributes to the decreased ability of elderly to properly accommodate with environmental stress (bacterial infection, inflammation . . . ). Muscle loss also participates to the reduction of physical activity and thus to the development of obesity, insulin resistance and osteoporosis in the elderly.

Although many mechanisms have been described concerning the etiology of this syndrome, the loss of muscle mass with age mainly results from a net protein loss and therefore an imbalance between muscle protein synthesis and degradation rates. During aging, resistance to certain anabolic factors appears, particularly a resistance to muscle protein synthesis stimulation by meal intake.

Food consumption in humans is characterized by its discontinuous nature, punctuated by the 2-3 main meals of the day, thus defining the circadian rhythm of nutritional status. This consumption profile includes several successive states: the postprandial state and the post-absorptive state. During the day, the various parameters of muscle protein metabolism fluctuate according to a cyclical profile involving nutritional status. During periods of fasting, the balance between synthesis and protein degradation is negative and causes a net loss of proteins compensated by a protein gain in response to food intake (periods of anabolism characterized by a net positive balance between protein synthesis and protein degradation rates). This alternation between catabolism phases and anabolism phases allows the maintenance of protein homeostasis. The reduction of muscle protein mass with age results from an imbalance between synthesis and protein degradation that could be established particularly in the postprandial period. Protein gain following the meal would no longer compensate for losses during the fasting period, and this permanent imbalance would eventually lead to a progressive loss of muscle proteins.

Thus, with meal intake, the rate of muscle protein synthesis physiologically increases. However, the regulating effect of food intake is blunted with age, participating in the development of sarcopenia.

This strong amino acidemia can be obtained through the consumption of animal proteins of high nutritional value, i.e. balanced amino acid composition and fast digestion. Nevertheless, epidemiological studies have shown that the consumption of proteins from animal sources, such as meat products, decreases sharply in the elderly. As a result, the production of novel foods for older people and the assessment of their nutritional quality could become major in the decades to come.

There is therefore still a need to develop compositions having improved physiological and/or pharmacological and/or therapeutic activities for the reduction of loss of muscle mass. It is accordingly one of the objects of the present invention to overcome or ameliorate at least one of the disadvantages of the prior art, or to provide a useful alternative.

### SUMMARY OF THE INVENTION

The present inventors surprisingly found that a combination of soluble fiber and protein reduces loss of muscle mass, preferably a combination of soluble fiber and pea protein.

Therefore, the present invention concerns a composition comprising soluble fiber and protein for reducing loss of muscle mass, preferably in sarcopenia conditions.

The present inventors have found that the present composition synergistically increase the nitrogen balance and the biological value.

In particular, consumption of pea protein when it is accompanied by soluble fibers, such as inulin, results in an increase in (skeletal) muscle mass, which is particularly evident compared to pea protein without accompanying soluble fibers.

Further, an increase in the systemic concentration of anti-inflammatory factors, such as interleukin-10, has been observed, without wishing to be bound by theory potentially signalling one of the mechanisms of action of pea protein with or without fibers on protein metabolism.

Despite the lower digestibility of pea protein, a significant effect of the type of protein has been shown for nitrogen balance, with a higher nitrogen balance observed when pea protein together with soluble fibers, such as inulin is consumed. Hence, despite a lower digestibility, the retention rate of pea protein is effective. This is also apparent from the biological value which is higher with pea protein and even more so when combined with soluble fibers, such as inulin.

It was further observed that muscle protein synthesis rates were increased at postprandial state compared to the post-absorptive state upon administration of pea proteins and that the presence of soluble fibers, such as inulin, in the diet is able to increase the potential for postprandial stimulation of food proteins on muscle anabolism, in particular in sarcopenic subjects. Accordingly, soluble fibers, such as inulin, allow increasing protein synthesis and muscle anabolism, in particular in combination with pea proteins. Hence, soluble fibers, such as inulin increase the effects of pea proteins on muscle anabolism and hence muscle mass.

Further, it was shown that mitochondrial density and enzyme activity were higher upon feeding pea protein and soluble fibers, such as inulin, indicating an improvement of muscle ATP production capacity.

The nitrogen balance is equal to nitrogen intake minus fecal and urinary nitrogen.

The nitrogen balance is measured by subtracting the nitrogen excreted with the nitrogen ingested. Urine and faeces are collected for 48 hours and the food consumption of the animals is measured. Then, the nitrogen concentration in urine and faeces is analysed and the amount of nitrogen ingested is calculated.

The nitrogen balance is calculated by: ingested nitrogen−excreted nitrogen; expressed in g/day.

The nitrogen content (for ingested and/or excreted nitrogen) can be determined by the Dumas method. (Dumas, A. Stickstoffbestimmung nach Dumas. Die Praxis des org.Chemikers (N-Determination according to Dumas), 41st ed.; Schrag: Nürnberg, Germany, 1962.)

Biological value (BV) is a measure of the proportion of absorbed protein from a food which becomes incorporated into the proteins of the organism's body. It captures how readily the digested protein can be used in protein synthesis in the cells of the organism. Proteins are the major source of nitrogen in food. BV assumes protein is the only source of nitrogen and measures the proportion of absorbed nitrogen that is retained in the body.

$$Biological value = \frac{Net Protein Utilization * 100}{True Nitrogen Digestibility}$$

The Biological value is expressed in %.

The Net protein utilization (NPU) corresponds to nitrogen retention or proportion of nitrogen intake that is retained in the body. It is determined by the following formula:

$$NPU = \frac{( ( NI ) - ( UN + FN ) + ( EFN + EUN ) )}{NI} * 100$$

NI=Nitrogen Intake; UN=Urinary Nitrogen; FN=Fecal Nitrogen; EFN=Endogenous Fecal Nitrogen; EUN=Endogenous Urinary Nitrogen

The net protein utilization is expressed in %.

The True Nitrogen Digestibility corresponds to the proportion of food nitrogen that is absorbed, as determined by the rat balance method. It is determined by the following formula:

$$True digestibility = \frac{NI - ( FN - EFN )}{NI} * 100$$

The true digestibility is expressed in %.

Another aspect of the invention also concerns a composition comprising, consisting essentially of, or consisting of soluble fiber and protein for use in preventing and/or reducing muscle loss.

Another aspect of the invention also concerns a composition comprising, consisting essentially of, or consisting of soluble fiber and protein for (therapeutic) use in maintaining or increasing the nitrogen balance.

Another aspect of the invention also concerns a composition comprising, consisting essentially of, or consisting of soluble fiber and protein for (therapeutic) use in maintaining or increasing the nitrogen balance in a subject having or being at risk of muscle loss.

Another aspect of the invention also concerns a composition comprising, consisting essentially of, or consisting of soluble fiber and protein for (therapeutic) use in maintaining or increasing the nitrogen balance in a subject having physical inactivity, chronic diseases like diabetes, obesity, sarcopenia, cachexia.

Another aspect of the invention also concerns a composition comprising, consisting essentially of, or consisting of soluble fiber and protein for (therapeutic) use in maintaining or increasing the net protein utilization.

Another aspect of the invention also concerns a composition comprising, consisting essentially of, or consisting of soluble fiber and protein for (therapeutic) use in maintaining or increasing the net protein utilization in a subject having or being at risk of muscle loss.

Another aspect of the invention also concerns a composition comprising, consisting essentially of, or consisting of soluble fiber and protein for (therapeutic) use in maintaining or increasing the net protein utilization in a subject having physical inactivity, chronic diseases like diabetes, obesity, sarcopenia, cachexia.

Another aspect of the invention also concerns a composition comprising, consisting essentially of, or consisting of soluble fiber and protein for (therapeutic) use in maintaining or increasing the biological value.

Another aspect of the invention also concerns a composition comprising, consisting essentially of, or consisting of soluble fiber and protein for (therapeutic) use in maintaining or increasing the biological value in a subject having or being at risk of muscle loss.

Another aspect of the invention also concerns a composition comprising, consisting essentially of, or consisting of soluble fiber and protein for (therapeutic) use in maintaining or increasing the biological value in a subject having physical inactivity, chronic diseases like diabetes, obesity, sarcopenia, cachexia.

Another aspect of the invention also concerns the (non-therapeutic) use of a composition comprising, consisting essentially of, or consisting of soluble fiber and protein for maintaining or increasing the nitrogen balance.

Another aspect of the invention also concerns the (non-therapeutic) use of a composition comprising, consisting essentially of, or consisting of soluble fiber and protein in maintaining or increasing the nitrogen balance in a subject having or being at risk of muscle loss.

Another aspect of the invention also concerns the (non-therapeutic) use of a composition comprising, consisting essentially of, or consisting of soluble fiber and protein in maintaining or increasing the nitrogen balance in a subject having physical inactivity, chronic diseases like diabetes, obesity, sarcopenia, cachexia.

Another aspect of the invention also concerns the (non-therapeutic) use of a composition comprising, consisting essentially of, or consisting of soluble fiber and protein in maintaining or increasing the net protein utilization.

Another aspect of the invention also concerns the (non-therapeutic) use of a composition comprising, consisting essentially of, or consisting of soluble fiber and protein in maintaining or increasing the net protein utilization in a subject having or being at risk of muscle loss.

Another aspect of the invention also concerns the (non-therapeutic) use of a composition comprising, consisting essentially of, or consisting of soluble fiber and protein in maintaining or increasing the net protein utilization in a subject having physical inactivity, chronic diseases like diabetes, obesity, sarcopenia, cachexia.

Another aspect of the invention also concerns the (non-therapeutic) use of a composition comprising, consisting essentially of, or consisting of soluble fiber and protein in maintaining or increasing the biological value.

Another aspect of the invention also concerns the (non-therapeutic) use of a composition comprising, consisting essentially of, or consisting of soluble fiber and protein in maintaining or increasing the biological value in a subject having or being at risk of muscle loss.

Another aspect of the invention also concerns the (non-therapeutic) use of a composition comprising, consisting essentially of, or consisting of soluble fiber and protein in maintaining or increasing the biological value in a subject having physical inactivity, chronic diseases like diabetes, obesity, sarcopenia, cachexia.

Another aspect of the invention also concerns a method for maintaining or increasing the nitrogen balance comprising administering a composition comprising, consisting essentially of, or consisting of soluble fiber and protein.

Another aspect of the invention also concerns a method for maintaining or increasing the nitrogen balance in a subject having or being at risk of muscle loss comprising administering a composition comprising, consisting essentially of, or consisting of soluble fiber and protein.

Another aspect of the invention also concerns a method for maintaining or increasing the nitrogen balance in a subject having physical inactivity, chronic diseases like diabetes, obesity, sarcopenia, cachexia comprising administering a composition comprising, consisting essentially of, or consisting of soluble fiber and protein.

Another aspect of the invention also concerns a method for maintaining or increasing the net protein utilization comprising administering a composition comprising, consisting essentially of, or consisting of soluble fiber and protein.

Another aspect of the invention also concerns a method for maintaining or increasing the net protein utilization in a subject having or being at risk of muscle loss comprising administering a composition comprising, consisting essentially of, or consisting of soluble fiber and protein.

Another aspect of the invention also concerns a method for maintaining or increasing the net protein utilization in a subject having physical inactivity, chronic diseases like diabetes, obesity, sarcopenia, cachexia comprising administering a composition comprising, consisting essentially of, or consisting of soluble fiber and protein.

Another aspect of the invention also concerns a method for maintaining or increasing the biological value comprising administering a composition comprising, consisting essentially of, or consisting of soluble fiber and protein.

Another aspect of the invention also concerns a method for maintaining or increasing the biological value in a subject having or being at risk of muscle loss comprising administering a composition comprising, consisting essentially of, or consisting of soluble fiber and protein.

Another aspect of the invention also concerns a method for maintaining or increasing the biological value in a subject having physical inactivity, chronic diseases like diabetes, obesity, sarcopenia, cachexia comprising administering a composition comprising, consisting essentially of, or consisting of soluble fiber and protein.

Another aspect of the invention also concerns a composition comprising, consisting essentially of, or consisting of soluble fiber and protein wherein the ratio soluble fiber/protein is ranging between 5%/95% to 75%/25% by weight, such as 5%/95% to 75%/25% by weight or 20%/80% by weight to 50%/50% by weight.

The present invention also encompasses a food or beverage product or a food supplement comprising between 0.1 and 40 g, such as between 5 and 40 g, such as between 5 and 30 g of a composition according to the present invention, per serving of the said food or beverage product or food supplement, or comprising between 5% and 75% by weight based on dry matter, preferably between 10% and 65%, of a composition according to the present invention.

The present invention also contemplates the use of a composition according to the present invention as a food additive in the production of a food or beverage product or a food supplement, which comprises between 0.1 and 40 g, such as between 5 and 40 g, such as between 5 and 30 g of said composition per serving of the said food or beverage product or food supplement, or comprising between 5% and 75% by weight based on dry matter, preferably between 10% and 65%, of a composition according to the present invention.

The present invention also encompasses a food or beverage product or a food supplement as described herein according to the invention for use in preventing and/or reducing muscle loss.

### DESCRIPTION OF THE FIGURES

FIG. 1 represents graphs plotting the effect of whey protein, pea protein and mixture of inulin and protein according to embodiment of the invention on nitrogen balance.

FIG. 2 represents graphs plotting the effect of whey protein, pea protein and mixture of inulin and protein according to embodiment of the invention on true nitrogen digestibility.

FIG. 3 represents graphs plotting the effect of whey protein, pea protein and mixture of inulin and protein according to embodiment of the invention on net protein utilization.

FIG. 4 represents graphs plotting the effect of whey protein, pea protein and mixture of inulin and protein according to embodiment of the invention on Biological value.

### DESCRIPTION OF THE INVENTION

Hereto, the present invention is in particular captured by any one or any combination of one or more of the below aspects and embodiments and numbered statements 1 to 43.

1. A composition comprising soluble fiber and protein for use in preventing and/or reducing loss of muscle mass, increasing nitrogen balance, increasing net protein utilization, and/or increasing biological value.

2. The composition according to statement 1, wherein said composition comprises at least 7 wt % by weight of soluble fiber based on dry matter.

3. The composition according to statement 1 or 2, wherein said composition comprises at least 10 wt % by weight of protein based on dry matter.

4. The composition according to any of statements 1 to 3, wherein the ratio of said soluble fiber to said protein is between 5/95 to 75/25 by weight.

5. The composition according to any of statements 1 to 4, wherein said soluble fiber is inulin.

6. The composition according to statement 5, wherein said inulin has an average degree of polymerization by number below 50.

7. The composition according to statement 5 or 6, wherein said inulin has an average degree of polymerization by number of at least 2.

8. The composition according to any of statements 5 to 7, wherein said inulin is obtained from a plant selected from the group comprising elecampane, dandelion, dahlia, wild yam, artichoke, Jerusalem artichokes, chicory jicama, burdock, onion, garlic, agave, yacón, banana, leek, asparagus, camas, or a mixture thereof.

9. The composition according to any of statements 1 to 8, wherein said protein is selected from animal protein, vegetable protein, alone or mixture thereof.

10. The composition according to statement 9, wherein said protein is preferably vegetable protein.

11. The composition according to statement 9 or 10, wherein said vegetable protein is pulse protein.

12. The composition according to any of statements 9 to 11, wherein said protein is pea protein.

13. The composition according to statement 9, wherein said protein is preferably animal protein.

14. The composition according to statement 13, wherein said animal protein is preferably whey protein.

15. The composition according to any of statements 1 to 14, wherein loss of muscle mass is caused by physical inactivity, chronic diseases like diabetes, obesity, sarcopenia, cachexia.

16. The composition according to statement 15, wherein loss of muscle mass is caused by sarcopenia.

17. The composition according to statement 15 or 16, wherein loss of muscle mass is caused by physical inactivity.

18. The composition according to any of statements 15 to 17, wherein loss of muscle mass is caused by diabetes.

19. The composition according to any of statements 15 to 18, wherein loss of muscle mass is caused by diabetes type I.

20. The composition according to one of statements 15 to 18, wherein loss of muscle mass is caused by diabetes type II.

21. The composition according to any of statements 15 to 18, wherein loss of muscle mass is caused by obesity.

22. The composition according to any of statements 1 to 21, wherein soluble fiber and protein have a synergistic effect.

23. The composition according to any of statements 1 to 22, wherein the composition is used in human food.

24. A composition comprising soluble fiber and protein, wherein the ratio of said soluble fiber to said protein is between 5/95 to 75/25 by weight.

25. The composition according to statement 24, wherein said composition comprises at least 7% by weight of soluble fiber based on dry matter.

26. The composition according to statement 24 or 25, wherein said composition comprises at least 10% by weight of protein based on dry matter.

27. The composition according to any of statements 24 to 26, wherein said soluble fiber is inulin.

28. The composition according to statement 27, wherein said inulin has an average degree of polymerization by number below 50.

29. The composition according to statement 27 or 28, wherein said inulin has an average degree of polymerization by number of at least 3.

30. The composition according to any of statements 27 to 29, wherein said inulin is obtained from a plant selected from the group comprising elecampane, dandelion, dahlia, wild yam, artichoke, Jerusalem artichokes, chicory, burdock, onion, garlic, agave, yacón, banana, leek, asparagus, camas, or a mixture thereof.

31. The composition according to statement 24, wherein said protein is selected from animal protein, vegetable protein, alone or mixture thereof.

32. The composition according to statement 31, wherein said protein is preferably vegetable protein.

33. The composition according to statement 31 or 32, wherein said vegetable protein is pulse protein.

34. The composition according to any one of statements 31 to 33, wherein said protein is pea protein.

35. The composition according to statement 31, wherein said protein is preferably animal protein.

36. The composition according to statement 35, wherein said animal protein is preferably whey protein.

37. The composition according to of statements 1 to 36, comprising at least 65% by weight based on dry matter, preferably at least 75%, more preferably at least 85%, even more preferably at least 95%, most preferably at least 98% of protein and soluble fiber.

38. A food or beverage product or food supplement comprising between 0.1 and 40 g of a composition according to any of statements 1 to 37, preferably per serving of the said food or beverage product or food supplement.

39. A food or beverage product or food supplement comprising between 5% and 75% by weight based on dry matter, preferably between 10% and 65%, of the composition according to any of statements 1 to 37.

40. A food or beverage product or food supplement according to statements 38 or 39, comprising between 0.5% and 30% by weight based on dry matter, of soluble fiber (i.e. the soluble fiber as defined in statements 1-37).

41. A food or beverage product or food supplement according to statements 38 or 39, comprising between 5% and 50% by weight based on dry matter, of protein (i.e. the protein as defined in statements 1-37).

42. A food or beverage product or food supplement according to statements 38 or 39, comprising between 0.5% and 30% by weight based on dry matter, of soluble fiber (i.e. the soluble fiber as defined in statements 1-37), and comprising between 5% and 50% by weight based on dry matter, of protein (i.e. the protein as defined in statements 1-37).

43. Use of a composition according to any of statements 1 to 37 as an ingredient, food additive, or feed additive in the production of a food or beverage product or a food supplement, optionally which comprises between 0.1 and 40 g of said composition per serving of the said food or beverage product or food supplement, or which comprises between 5% and 75% by weight based on dry matter, preferably between 10% and 65%, of said composition.

Before the present method of the invention is described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

The following detailed description, therefore, is not to be taken in a limiting sense, and the scope of the present invention is defined by the appended claims.

As used herein, the expression “%” refers to “% by weight expressed on dry matter”. The % can be calculated on the total composition according to the present invention. Alternatively, the % can be calculated from the ratio between two or more compounds of a mixture.

The present invention relates to a composition comprising soluble fiber and protein, such as a composition comprising soluble fiber and protein for use in reducing loss of muscle mass.

In certain embodiments, said composition comprises at least 7 wt % by weight of soluble fiber, preferably 10 wt %, more preferably 25 wt %, even more preferably 45 wt % of soluble fiber.

In certain embodiments, said composition comprises at least 10 wt % by weight of soluble fiber, preferably 25 wt %, more preferably 50 wt %, even more preferably 60 wt % of protein.

In certain embodiments, said composition comprises at least 5% by weight of soluble fiber, based on the total amount of protein and soluble fiber.

In certain embodiments, said composition comprises at most 75% by weight of soluble fiber, based on the total amount of protein and soluble fiber.

In certain embodiments, said composition comprises at least 25% by weight of protein, based on the total amount of protein and fiber.

In certain embodiments, said composition comprises at most 95% by weight of protein, based on the total amount of protein and fiber.

In certain embodiments, said composition comprises at least 5% by weight of soluble fiber, based on dry matter.

In certain embodiments, said composition comprises at most 75% by weight of soluble fiber, based on dry matter.

In certain embodiments, said composition comprises at least 25% by weight of protein, based on dry matter.

In certain embodiments, said composition comprises at most 95% by weight of protein, based on dry matter.

In certain embodiments, said composition has a weight ratio soluble fiber/protein comprises between 5/95 and 75/25, preferably 20/80 and 75/25, more preferably 25/75 and 50/50.

In certain embodiments, said composition comprises at least 65% by weight based on dry matter, preferably at least 75%, more preferably at least 85%, even more preferably at least 95%, most preferably at least 98% of protein and soluble fiber.

In the present invention, the term “soluble fiber” denotes soluble dietary fibers. Said fibers denote an entire series of different compounds which have the common property that they cannot be broken down by the digestive enzymes of human beings. Almost all dietary fibers are carbohydrate polymers.

Preferably, said soluble fiber of vegetable origin is chosen from the group consisting of fructans, including fructooligosaccharides (FOSs) and inulin, glucooligosaccharides (GOSs), arabinoxylan (AX), arabinoxylan-ioligosaccharides (AXOS), isomaltooligosaccharides (IMOs), trans-galactooligosaccharides (TOSs), pyrodextrins, polydextrose, branched maltodextrins, indigestible dextrins and soluble oligosaccharides derived from oleaginous plants or protein-producing plants.

The term “soluble fiber” is intended to mean fibers soluble in water. The fibers can be assayed according to various AOAC methods. By way of example, mention may be made of AOAC methods 997.08 and 999.03 for fructans, FOSs and inulin, AOAC method 2000.11 for polydextrose, AOAC method 2001.03 for assaying the fibers contained in branched maltodextrins and indigestible dextrins, or AOAC method 2001.02 for GOSs and also soluble oligosaccharides derived from oleaginous plants or protein-producing plants. Among the soluble oligosaccharides derived from oleaginous plants or protein-producing plants, mention may be made of soya, rapeseed or pea oligosaccharides.

According to a preferred embodiment of the invention, the composition comprises inulin and protein.

According to a preferred embodiment of the invention, the composition comprises soluble fiber and pea protein.

According to a preferred embodiment of the invention, the composition comprises inulin and pea protein.

According to a preferred embodiment of the invention, the composition comprises inulin and protein for use in reducing loss of muscle mass.

According to a preferred embodiment of the invention, the composition comprises soluble fiber and pea protein for use in reducing loss of muscle mass.

According to a preferred embodiment of the invention, the composition comprises inulin and pea protein for use in reducing loss of muscle mass.

In certain embodiments, the protein, such as pea protein, is a protein extract, such as pea protein extract. The extract preferably comprises at least 80% by weight based on dry matter of protein, such as pea protein, preferably at least 85%, such as at least 90%.

In certain embodiments, the soluble fiber, such as inulin, is a soluble fiber extract, such as inulin extract. The extract preferably comprises at least 80% by weight based on dry matter of soluble fiber, such as inulin, preferably at least 85%, more preferably at least 90%, such as at least 95%.

As used herein, the term “inulin” refers to a mixture of oligo- and/or polysaccharides of fructose which may have a terminal glucose. Inulins belong to a class of fibers known as fructans. In an embodiment, Inulin can be represented, depending from the terminal carbohydrate unit, by the general formulae GFn and Fm, wherein G represents a glucose unit, F represents a fructose unit, n is an integer representing the number of fructose units linked to the terminal glucose unit, and m is an integer representing the number of fructose units linked to each other in the carbohydrate chain. Inulins for use in the present invention encompasses inulins with a terminal glucose which are also referred as alpha-D-glucopyranosyl-[beta-D-fructofuranosyl](n-1)-D-fructofuranosides, as well as inulins without glucose which are also referred as beta-D-fructopyranosyl-[D-fructofuranosyl](n-1)-D-fructofuranosides. Inulins for use in the present invention can also encompass the hydrolysis products of inulins such as oligofructoses, which are fructose oligomers with a degree of polymerization (DP) of ≤20, and they can also encompass fructose oligomers ending with a terminal glucose with a DP of 3-5 synthesized from saccharose. Suitable oligosaccharide chains of inulin from plant origin for use in the invention can have a degree of polymerization (DP) ranging from 3 to about 100. Inulin can be a liquid or a powder product.

As used herein, the terms “degree of polymerization” or “(DP)” relates to the number of monosaccharide residues present in an oligo- or polysaccharide. Often also the parameter average degree of polymerization is used. The degree of polymerization is a measure of molecular weight (MW). The DP can be calculated as the ratio of the total MW of the polymer or oligomer and the MW of the repeating units.

In a particular embodiment, the composition comprises inulin and protein for use in reducing loss of muscle mass, inulin has an average DP by number below 50, for example between 2 and 50, for example between 2 and 40, for example between 2 and 30, for example between 5 and 30, for example between 5 and 20, for example between 5 and 15, and for example about 10.

In an embodiment, inulin for use in the composition can originate from or be isolated or obtained from any natural source of inulin known to date, or can be enzymatically synthesized from saccharose, or can be a commercially available inulin. In an embodiment, inulin originates from or is isolated from elecampane, dandelion, dahlia, wild yam, artichoke, Jerusalem artichokes, chicory, jicama, burdock, onion, garlic, agave, yacón, banana, leek, asparagus or camas. In an embodiment, inulin is a (largely) linear fiber. Preferably, inulin originates from, or is isolated from chicory or Jerusalem artichokes. Suitable commercial inulin for use in the invention can be selected from the group comprising Fibruline® Instant, Fibruline® XL, Fibruline® DS, Fibruline® S20, Fibrulose® F97, . . . (Cosucra Group Warcoing SA, Belgium), Frutafit® IQ, Frutafit® HD, Frutafit® TEX, Frutafit® CLR, Frutafit® L90, Frutafit® L85, . . . (Sensus, the Netherlands), Orafti® ST, Orafti® GR, Orafti® LGI, Orafti® HSI, Orafti® P95, Orafti® L85, Orafti® L60, Orafti® synergy1, Orafti® HP, . . . (Beneo-Orafti, Belgium), Actilight® 950P, Actilight® 950S, Actilight® 850S, . . . (Syral, France).

In a preferred embodiment, inulin for use in the composition originates from chicory or Jerusalem artichokes and has an average DP between 6 and 25.

In a preferred embodiment, the composition comprises soluble fiber and vegetable protein for use in reducing loss of muscle mass.

In the present invention, the term “vegetable protein” preferably comprises all proteins derived from cereals, oleaginous plants, tuberous plants and pulse and can be used alone or as a mixture.

In the present invention, the term “cereals” is intended to mean cultivated plants of the grass family producing edible seeds. Non-limiting examples are wheat, oats, rye, barley, corn, sorghum, millet or rice. The cereals are often milled in the form of flour but are also available as grains and sometimes in whole-plant form (fodders).

In the present invention, the term “oleaginous plants” denotes plants cultivated specifically for their seeds or their fruits rich in fats, from which oil for dietary, energy or industrial use is extracted. Non-limiting examples are rapeseed, groundnut, sunflower, soybean, sesame and the castor oil plant.

In the present invention, the term “tuberous plants” is intended to mean all plants comprising storage organs, which are generally underground, which ensure plant survival during the winter season and often plant multiplication by the vegetative process. These organs are bulging owing to the accumulation of storage substances. The organs transformed into tubers may be:
the root: Non-limiting examples are carrot, parsnip, cassava, konjac,
the rhizome: Non-limiting examples are potato, Jerusalem artichoke, Japanese artichoke, sweet potato,
the base of the stalk (more specifically the hypocotyl): Non-limiting examples are kohlrabi, celeriac,
the root+hypocotyl combination: Non-limiting examples are beetroot, radish.

In a preferred embodiment, the composition comprises soluble fiber and pulse protein for use in reducing loss of muscle mass.

In a preferred embodiment, the composition comprises inulin and pulse protein for use in reducing loss of muscle mass.

In the present invention, the term “pulses” is intended to mean the dried seeds of legumes. The four most common pulses are beans, chickpeas, lentils and peas. Lentils, as Lens culinaris, include for example: Beluga Lentils, Brown Lentils, French Green Lentils, Green Lentils and Red Lentils. Beans, as Phaseolus vulgaris, include for example: Adzuki Beans, Anasazi Beans, Appaloosa Beans, Baby Lima Beans, Black Calypso Beans, Black Turtle Beans, Dark Red Kidney Beans, Great Northern Beans, Jacob's Cattle Trout Beans, Large Faba Beans, Large Lima Beans, Mung Beans, Pink Beans, Pinto Beans, Romano Beans, Scarlet Runner Beans, Tongue of Fire, White Kidney Beans and White Navy Beans. Peas, as Pisum sativum include for example: Black-Eyed Peas, Green Peas, Marrowfat Peas, Pigeon Peas, Yellow Peas and Yellow-Eyed Peas. Chickpeas, as Cicer arietinum, include for example: Chickpea and Kabuli.

In a preferred embodiment, the composition comprises soluble fiber and pea protein for use in reducing loss of muscle mass.

In a most preferred embodiment, the composition comprises inulin and pea protein for use in reducing loss of muscle mass.

In a most preferred embodiment, pea protein is used. Dry peas contain 20-30% lysine rich proteins. Pea proteins are mainly storage protein comprised of albumins and two globulins, legumin and vicilin which proteins may relatively easily solubilized and isolated. In addition, these proteins are characterized by high lysine content, which is deficient in many other proteins of plant origin. Solubility profile of pea protein isolates is similar to other legume proteins and is characterized by high solubility at alkaline pH-s, a minimum solubility at isoelectric point and moderate solubility in acidic medium. They are characterized by a relatively good emulsifying activity.

Pea protein can be prepared in three forms: pea flour, pea protein concentrate, and pea protein isolate. Pea flour is produced by dry milling of dehulled peas. Pea protein concentrate can be produced through the acid leaching procedure traditionally used to produce soy protein concentrates, but it is more economical to use dry separation methods. Pea protein isolate is produced by wet processing methods.

Proteins and starches within non-oilseed legumes such as peas can be efficiently fractionated by dry milling and air classification. Fine grinding results in flours containing populations of particles differentiated by size and density. Air classification of such flours separates the protein (fine fraction) from the starch (coarse fraction). In this dry process, whole or dehulled peas are pin milled to a very fine flour. During milling starch granules remain relatively intact, while the protein matrix is broken down into fine particles. Care must be used in milling to avoid damage to starch granules. The flour is air classified in a spiral air stream into a fine fraction containing approximately 75% of the total protein, but only 25% of the total mass; and a coarse fraction, containing most of the starch granules. The starch and protein fractions are separated in the air classifier based on their differential mass and size. The coarse fraction can be centrifugally separated from the fine fraction and carried into a coarse fraction duct. The fine fraction is carried with the air into the air cyclone. After milling, some protein bodies still adhere to the starch granules and some starch is still imbedded in the protein matrix. The level of adhering protein bodies and agglomerated starch and protein can be reduced by repeated pin milling and air classification. With such a double pass process, a yield of 33-35% total protein fraction (protein content of 56%, dry basis, N×6.25) was obtained for peas with an original protein content of about 25%.

Highly concentrated protein fractions (protein isolates) and protein concentrates from pea can be prepared by wet processing. The protein separation has been mainly based on solubilization of protein followed by isoelectric precipitation for subsequent recovery.

The isoelectric precipitation process for protein isolate production involves milling of the peas, followed by solubilization of the proteins in water, alkali, or acid; then centrifuging to remove the insoluble components. The solubilized proteins are then precipitated at their isoelectric pH. The precipitated protein curd is collected by centrifugation or sieving. The curd is dried as such to yield isoelectric protein isolate or neutralized and dried to yield a cationic-protein isolate.

Protein isolate have a total protein content (expressed in N 6.25) of at least 70% based on the dry matter, at least 75%, at least 80%, at least 85%, at least 90%, for example between 70% and 99%, preferably between 80% and 95%, more preferably between 80% and 90%. In a preferred embodiment, pea protein isolate is used.

In a preferred embodiment, non-hydrolysed vegetable protein is used. The term “non-hydrolysed protein” is equivalent to an “intact” protein, meaning that the protein has not been subjected to a hydrolysis process. However, minor amounts of hydrolysed proteins may be present in the source of non-hydrolysed proteins, or may be added to the formulation, such as additional amino acids, such as, for example leucine, isoleucine, glutamine, arginine, or dipeptides and the like. According to another embodiment, intact protein may only possess a degree of hydrolysis (DH) of 5% of lower, preferably 4%, 3%, 2%, 1% or lower.

In an embodiment, the composition comprises soluble fiber and animal protein for use in reducing loss of muscle mass and may be selected from whey protein, casein or egg protein, alone or in combination.

In a preferred embodiment, the composition comprises soluble fiber and whey protein for use in reducing loss of muscle mass.

As such, the invention also encompasses a composition consisting of soluble fiber and protein and its use for reducing loss of muscle mass, such as physical inactivity, chronic disease like diabetes, obesity and sarcopenia. Preferably the present composition is used for reducing loss in muscle mass in sarcopenia.

As used herein, the term “synergism” or “synergy” refers to two or more agents working together to produce a result not obtainable by any of the agents independently. This term is used to describe a situation where different entities cooperate advantageously for a final outcome. As used herein the terms “synergistic amounts” or “synergetic amounts” refer to amounts of inulin and protein which together achieve a more pronounced effect than each alone or may even achieve an effect greater than for the sum of each alone.

The inventors surprisingly found that soluble fiber and protein have synergistic effects in reducing loss of muscle mass, in particular in sarcopenia. In a particular embodiment, the present invention provides a composition comprising soluble fiber and protein, wherein said soluble fiber and protein are present in synergistic amounts. The compositions therefore offer great potential to better increase the nitrogen balance and the biological value.

The present invention relates to a composition comprising soluble fiber and protein, wherein said the ratio of said soluble fiber to said protein is between 5/95 to 75/25 by weight, preferably 20/80 and 75/25, more preferably 25/75 and 50/50.

In certain embodiments, said composition comprises at least 7 wt % by weight of soluble fiber, preferably 10 wt %, more preferably 25 wt %, even more preferably 45 wt % of soluble fiber.

In certain embodiments, said composition comprises at least 10 wt % by weight of soluble fiber, preferably 25 wt %, more preferably 50 wt %, even more preferably 60 wt % of protein.

In an embodiment, the composition comprises inulin and pea protein, wherein the ratio of said soluble fiber to said protein is between 5/95 to 50/50 by weight.

In a particular embodiment, the composition comprises inulin and protein, inulin has an average DP by number below 50, for example between 2 and 50, for example between 2 and 40, for example between 2 and 30, for example between 5 and 30, for example between 5 and 20, for example between 5 and 15, and for example about 10.

In a preferred embodiment, inulin in the composition originates from chicory or Jerusalem artichokes and has an average DP between 6 and 25.

In a preferred embodiment, the composition comprises soluble fiber and vegetable protein, wherein the ratio of said soluble fiber to said protein is between 20/80 to 50/50 by weight.

In a preferred embodiment, the composition comprises soluble fiber and pulse protein, wherein the ratio of said soluble fiber to said protein is between 5/95 to 75/25 by weight.

In a preferred embodiment, the composition comprises inulin and pulse protein, wherein the ratio of said soluble fiber to said protein is between 5/95 to 75/25 by weight.

In a preferred embodiment, the composition comprises soluble fiber and pea protein, wherein the ratio of said soluble fiber to said protein is between 5/95 to 75/25 by weight.

In a most preferred embodiment, the composition comprises inulin and pea protein, wherein the ratio of said soluble fiber to said protein is between 5/95 to 75/25 by weight.

In an embodiment, the composition comprises soluble fiber and animal protein, wherein the ratio of said soluble fiber to said protein is between 5/95 to 75/25 by weight and may be selected from whey protein, casein or egg protein, alone or in combination.

In a preferred embodiment, the composition comprises soluble fiber and whey protein, wherein the ratio of said soluble fiber to said protein is between 5/95 to 75/25 by weight.

The present invention therefore also concerns the use of a composition according to the invention as described herein comprising, consisting essentially of, or consisting of soluble fiber and protein for the preparation of a food, beverage, or (food/beverage) supplement and/or a medicament for reducing loss of muscle mass preferably in case of sarcopenia (the food, beverage, or (food/beverage supplement) may be a medicament). The present invention also concerns a method for reducing loss of muscle mass comprising the administration of a physiologically or therapeutically effective amount of a composition according to the invention as described herein comprising, consisting, or consisting essentially of soluble fiber and protein in an individual in need thereof.

The present invention relates to a composition according to the invention as described herein comprising, consisting essentially of, or consisting of soluble fiber and protein and its use for reducing loss in muscle mass. The present invention also relates to a composition comprising, consisting essentially of, or consisting of a soluble fiber and a protein, wherein the ratio of said soluble fiber to said protein is between 5/95 to 75/25 by weight. As used herein, the term “comprising” means that the composition contains at least soluble fiber and protein. Additional compounds, ingredients, products may or may not be present in such composition. Non-limiting examples of additional ingredients include other fermentable fibers, carbohydrates, proteins, fats, minerals, vitamins.

As used herein the term “therapeutically effective amount” of said above-described composition relates to the amount or quantity of said composition required to achieve the desired therapeutic and/or prophylactic effect. Effective amounts may be measured and expressed in g/day.

As used herein the term “physiologically effective amount” of said above-described composition relates to the amount or quantity of said composition required to achieve the desired physiological effect. Effective amounts may be measured and expressed in g/day.

The composition according to the invention can be supplemented to food, for instance to functional food, dietetic food and/or food supplements, as a food additive, in particular a functional food additive. The present invention also encompasses a method for preparing a food product or beverage or food supplement comprising the steps of: (a) providing a composition according to the present invention, and (b) formulating said composition into a food product, a feed product, a beverage or a supplement.

The present invention also concerns a food product containing the composition according to the present invention, as well as a feed product containing the same composition, a beverage containing the same and a food supplement containing the same.

The composition of the invention may be used as a food additive in the production of a food or beverage, or as a basis for a food supplement. In an embodiment, the food or beverage or supplement comprises between 0.1 and 40 g of the composition according to the present invention per serving of said food or beverage or supplement. In a preferred embodiment, the food or beverage or supplement comprises between 0.5 and 20 g of the composition according to the present invention per serving of said food or beverage or supplement. In an even more preferred embodiment, the food or beverage or supplement comprises between 1 and 10 g of the composition according to the present invention per serving of said food or beverage or supplement.

It will be understood that the food, beverage, or supplement may relate to a food, beverage, or supplement which is ready for consumption, but may also relate to a food, beverage, or supplement fraction, concentrate or the like, which may need to be reconstituted or otherwise processed in order to obtain a food, beverage, or supplement which is ready for consumption (e.g. instant powders, doughs, etc.)

For pharmaceutical use, the compositions of the invention may be formulated as a pharmaceutical preparation comprising soluble fiber and protein and at least one pharmaceutically acceptable carrier, diluent or excipient and/or adjuvant, and optionally one or more further pharmaceutically active compounds. Such a formulation may be in a form suitable for oral administration.

In an embodiment, the present composition can optionally be combined with at least one pharmaceutically acceptable carrier for oral administration. When combined with a carrier, the weight percent of the carrier on the total composition can be between 1 and 85%. Typical carriers are food and water. If soluble fiber is used, the combination of an aqueous carrier and the soluble fiber will be a solution. If insoluble fiber is used, the combination of an aqueous carrier and the fiber will be a suspension. The compositions can include an inert diluent or an edible carrier. They may be enclosed in gelatin capsules or compressed into tablets. For the purpose of oral therapeutic administration, the composition can be incorporated with excipients and used in the form of tablets, troches, suppositories or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. The composition can be administered as a component of an elixir, suspension, syrup, wafer, chewing gum or the like. A syrup may contain, in addition to the active compounds, sucrose as a sweetening agent and certain preservatives, dyes and colorings and flavors. The composition can also be mixed with other active materials which do not impair the desired action, or with materials that supplement the desired action.

The pharmaceutical preparations are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

The present composition will generally be administered in an effective amount, which, upon suitable administration, is sufficient to achieve the desired physiological, therapeutic and/or prophylactic effect in the individual to which it is administered. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the composition.

The invention will now be illustrated by means of the following examples, which do not limit the scope of the invention in any way.

### EXAMPLES

### 1. Materials and Methods

### 1.1. Products

The standard diet is composed of 6% fat (Soybean oil), 80% carbohydrates (10% sucrose, 40.6% of starch, 15.5% of maltodextrin and 8.3% of cellulose) and 14% proteins in the form of casein (7.5% of moisture).

For comparison, 2 types of proteins were used: whey and pea protein.

The pea protein source in this trial was Pisane® C9 (COSUCRA groupe Warcoing SA, Belgium), which is a pea protein isolate with a dry matter of 95 wt.+/−2% and a protein content of 86+/−2 wt. %

The inulin source used in this trial was Fibruline® Instant (COSUCRA groupe Warcoing SA, Belgium), which is a chicory inulin with a DP ranging from 2 to 60 and an average DP (by number) of about 10. Fibruline® Instant was a powder with a dry matter of 96% and contained, on dry matter, 90% of inulin.

### 1.2. Subjects

Rats were delivered to our animal facilities 2 weeks before the start of the study in order to acclimate to their new environment. Throughout the study, the rats were placed in individual cages in a controlled environment, i.e. light (12 h light, 12 h night), temperature (20-22° C.) and hygrometry (50-60% humidity). The rats were fed with ceramic feeders designed to prevent food losses. Animals and food were weighed once a week. Water was provided ad libitum.

After 2 weeks of acclimatization, rats were weighed, and their body composition was measured using an Echo-MRI® system on the principle of nuclear magnetic resonance to measure the fat mass and lean mass. The rats were then divided according to body weight, lean mass and body fat in 5 equivalent groups.

### 1.3. Experimental Design

These groups of 20 rats were fed with 5 different diets for 16 weeks:
either with a standard diet (6% fat, 80% carbohydrates and 14% proteins in the form of casein; all based on dry weight)
either with a diet containing dietary fast protein, i.e. whey protein (6% fat, 80% carbohydrates and 14% proteins in the form of whey; all based on dry weight)
either with a diet containing whey protein with inulin (6% fat, 72.5% carbohydrates, 7.5% inulin and 14% proteins in the form of whey; all based on dry weight)
either with a diet containing pea protein (6% fat, 80% carbohydrates and 14% protein in the form of pea protein; all based on dry weight)
either with a diet containing pea protein with inulin (6% fat, 72.5% carbohydrates, 7.5% inulin and 14% proteins in the form of pea protein; all based on dry weight)

Five days before the end of the 16 weeks period, the rats were placed in a metabolic cage so that an accurate and complete measure of food intake (drinking water and food) and excreta (urine and feces) could be carried out. The analysis of nitrogen content in feces and urine was then determined by combustion method according to the Dumas method. (Dumas, A. Stickstoffbestimmung nach Dumas. Die Praxis des org.Chemikers (N-Determination according to Dumas), 41st ed.; Schrag: Nürnberg, Germany, 1962).

At the end of the period, 72 animals were still alive and distributed as described in Table 1.

TABLE 1
Number of remaining animals at the
end of the experimental protocol.

| Diet | Remaining rats (/20) |
|---|---|
| Control | 14 |
| Whey | 12 |
| Whey + inulin | 16 |
| Pea | 16 |
| Pea + inulin | 14 |

At the end of the 16-week period, rats were fasted for 12 hours. Half of the rats in each group were force-fed with a solution rich in carbohydrates and amino acids (gavage). All rats then received a stable isotopic tracer infusion (13C-valine) 50 minutes before being sacrificed to measure protein synthesis rate. After sacrifice, blood, subcutaneous and perirenal adipose tissues, heart, liver, kidneys, duodenum, jejunum, ileum and different types of skeletal muscles of the hindlimb (soleus, plantaris, tibialis anterior, gastrocnemius and quadriceps) were collected.

### 1.4. Statistical Analysis

The results are presented as means±standard deviations to the mean (SEM). An analysis of variance (ANOVA) was used to compare the groups with each other (effect of the diet), followed by a posterior Fischer test. Statistical analysis was performed using the software Statview and NCSS. The differences were considered significant when p<0.05. NS means no significant difference (ANOVA p>0.05). A two-factor ANOVA was also used to explore the protein type effect and the fiber effect. An ANOVA on repeated measurements followed by a posteriori Tukey-Kramer test has been used to study the time changes in weight, body composition and food intake.

### 2. Results

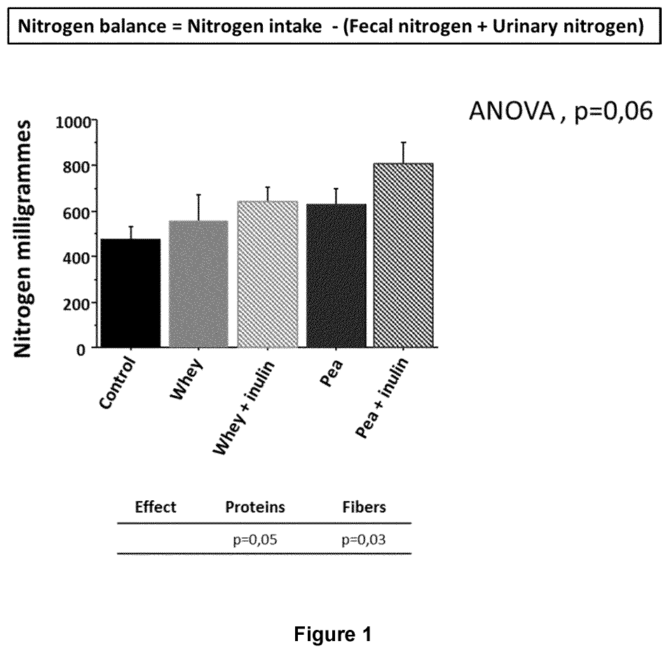

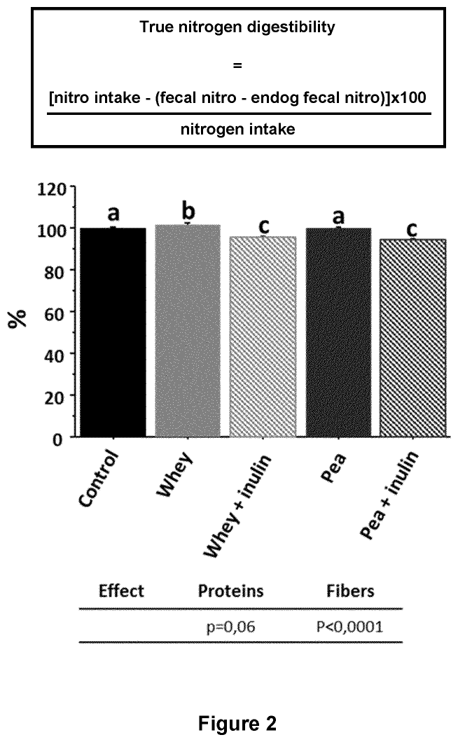

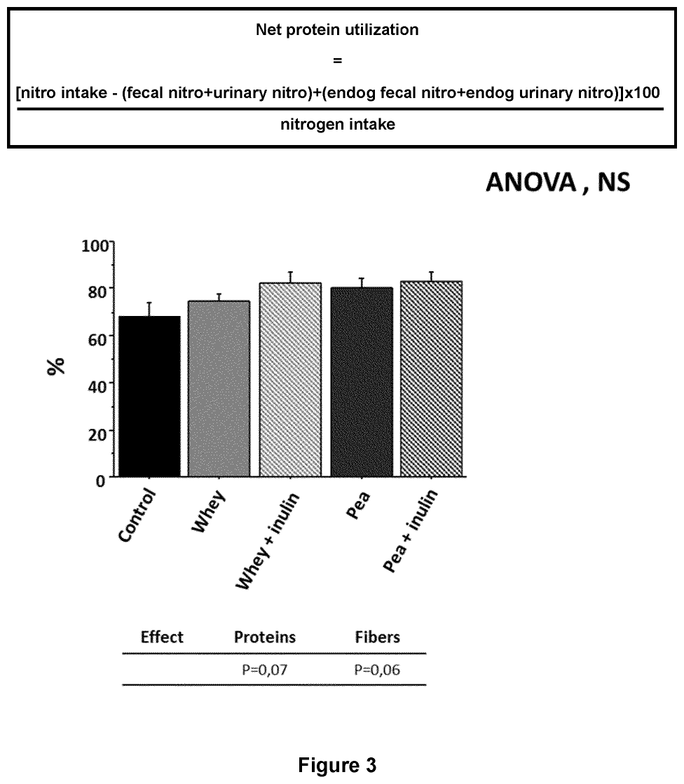

A significant effect of the type of proteins or the presence of dietary fiber in the diet was observed for the nitrogen balance (2-factor ANOVA—FIG. 1-4). This effect was partly explained by a higher nitrogen balance in the fiber consuming groups, especially in the presence of the pea protein. The true nitrogen digestibility was highest in rats fed with the whey diet compared to those measured in rats fed with the other diets while it was lowest in rats fed diets containing inulin (p<0.05) (FIG. 2). The digestion rate of a protein can be reduced but its use by the organism can be optimized due to a balanced amino acid composition with respect to the needs. Also, although the rate of digestion is reduced, the lower nitrogen excretion in urine reflects a decrease in the oxidation rate (and therefore irreversible destruction) of amino acids. In the end, the balance is better (less digestion but more efficient use).

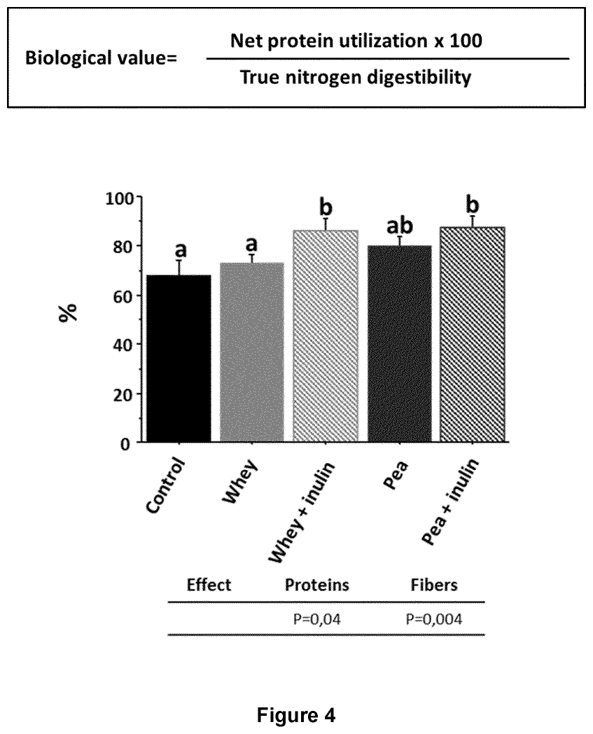

The biological value was higher in rats that consumed diets containing inulin compared to those receiving control and whey diets (FIG. 4). Likewise, biological value tend to increase in rats fed pea protein.

1. A composition comprising inulin and protein for use in preventing and/or reducing loss of muscle mass.

2. A composition comprising inulin and protein for use in increasing the biological value, the net protein utilization, and/or the nitrogen balance.

3. The composition according to claim 1, wherein the ratio of said inulin to said protein is between 5/95 to 75/25 by weight.

4. The composition according to claim 1, wherein said inulin is obtained from a plant selected from the group comprising elecampane, dandelion, dahlia, wild yam, artichoke, Jerusalem artichokes, chicory, burdock, onion, garlic, agave, yacón, banana, leek, asparagus, camas, or a mixture thereof.

5. The composition according to claim 1, wherein said protein is selected from animal protein, vegetable protein, alone or mixture thereof.

6. The composition according to claim 5, wherein said protein is vegetable protein, more preferably pulse protein, even more preferably pea protein.

7. The composition according to claim 5, wherein said protein is animal protein, more preferably whey protein.

8. The composition according to claim 1, wherein loss of muscle mass is caused by physical inactivity, chronic diseases like diabetes, obesity, or sarcopenia.

9. The composition according to claim 8, wherein loss of muscle mass is caused by sarcopenia.

10. The composition according to claim 1, wherein inulin and protein have a synergistic effect.

11. A composition comprising inulin and protein, wherein the ratio of said inulin to said protein is between 5/95 to 75/25 by weight, and comprising at least 65% by weight based on dry matter, preferably at least 75%, more preferably at least 85%, even more preferably at least 95%, most preferably at least 98% of protein and inulin.

12. A food or beverage product or food supplement comprising between 0.1 and 40 g of a composition according to claim 1, per serving of the said food or beverage product or food supplement, and/or comprising between 5% and 75% by weight based on dry matter, preferably between 10% and 65%, of the composition according to claim 1.

13. Use of a composition according to claim 1 as a food or feed additive in the production of a food or beverage product or a food supplement, which comprises between 0.1 and 40 g of said composition per serving of the said food or beverage product or food supplement, and/or comprising between 5% and 75% by weight based on dry matter, preferably between 10% and 65%, of said composition.

14. A food or beverage product or food supplement comprising between 0.1 and 40 g of a composition according to claim 11 per serving of the said food or beverage product or food supplement, and/or comprising between 5% and 75% by weight based on dry matter, preferably between 10% and 65%, of the composition according to claim 11.
